(19) United States
(12) Patent Application Publication
(10) Pub. No.: US 2010/0189211 A1
(43) Pub. Date: Jul. 29, 2010

(54) X-RAY SOUCE FOR MEASURING RADIATION

(75) Inventor: Rolf Karl Otto Behling, Norderstedt (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/668,044

(22) PCT Filed: Jul. 7, 2008
(86) PCT No.: PCT/IB08/52718

§ 371 Date: Jan. 7, 2010

(30) Foreign Application Priority Data
Jul. 11, 2007 (EP) 07112285.7

(51) Int. Cl. A61B 6/03; H01J 35/10; H01J 35/08; G01N 23/04

(52) U.S. Cl. 378/4; 378/124; 378/57; 378/62; 378/154

(57) ABSTRACT

Cone-beamCT scanners with large detector arrays suffer from increased scatter radiation. This radiation may cause severe image artefacts. According to an exemplary embodiment of the present invention, an examination apparatus is provided which directly measures the scatter radiation. The measurement is performed by utilizing an X-raytube with an anode disk (500) comprising a slit (510) which is positioned in a 5 target area (512) of the anode disk. The slit opening is adapted to be penetrated at least partially by the electron beam (580) from the cathode of the x-raytube to alternatingly create a secondary source of X-rays (555) from a second anode (550), whereby the secondary source is located outside the focus area of the anti-scatter grid of the X-raydetector. Cone-beamCT scanners may also suffer from cone beam artifacts. An X-10 raytube is described, which helps measuring an additional set of scan data.

The invention relates to the field of tomographic imaging. In particular, the invention relates to an anode disk, an x-ray tube, an examination apparatus for examination of an object of interest, to a method of examination of an object of interest, a computer-readable medium and a program element.

Scattered radiation may deteriorate the image signal especially for large detectors, as it overlaps with the direct radiation. The scattered radiation may cause severe image artefacts. In order to reduce image artefacts resulting from scatter radiation, two-dimensional or one-dimensional anti-scatter-grids (ASG) may be used. An ASG limits the space angle, from which scattered radiation may reach cells of a detector unit, and therefore, improves the ratio of scattered radiation to direct radiation. Scattered radiation has a low spatial frequency compared to direct radiation and thus may be well distinguished.

For measurement of the scattered radiation, a focus of the x-ray source would need to be moved out of a focal range defined by the ASG. The focal range is the range from where the focus could utmost send direct radiation to the detector unit. The movement for measuring the scattered radiation is limited by still having the option to measure the spatial patterns of the scattered radiation on the detector unit. The negative effect of the additional dose to a human body, when used in a diagnostic CT system, is usually more than offset by the increased image quality and diagnostic value.

It may be desirable to temporally displace an anodes focal spot of the x-ray source to the periphery of the focal range of a ASG.

Beside the said scattering problem, it may be desirable to measure an additional set of scan data during different sequences for different purposes, as i.e. to measure cone-beam artifacts.

According to an exemplary embodiment of the present invention, an anode disk is provided for later measuring scattered radiation of a rotating anode x-ray tube. The anode disk comprises a slit positioned in a target area of the anode disk. The slit opening is adapted to be penetrated at least partially by an electron beam of a cathode of the x-ray tube. In one aspect of the embodiment, the width of the slit is in a range between 0.9 mm and 3 mm. A slit width of 2 mm has been proved as good choice. In a further embodiment, the anode disk comprises a plurality of slits. The slit is cut into the anode disk at the focal track of the rotating anode disk.

In another aspect the anode disk further comprises a cavity. The cavity incorporates the slit and may be shaped such as to avoid transition effects when the electron beam penetrates trough the anode disk, precisely the slit. In other words, electrons fall into the notch before passing the slit, and thus may not leave the X-ray source. For a plurality of slits, the same number of notches may be provided. In yet another aspect of the invention, the missing anode material due to existence of slit and notch or a plurality of the said, may be balanced with additional material on the side of the anode disk facing away from the cathode.

Scatter data may be acquired when the focus has been moved due to anode disk rotation relative to the anode disk trough the slit which is in the target area of the incident electron beam (focal track of the anode disk) of the cathode. Thus, by moving the anode disk, two different data sets may be acquired, mainly the image data from direct radiation and, at a position of the disk where the slit is penetrated by the electron beam, the scatter data. This scatter data may be used for image correction.

In a further embodiment, an x-ray tube comprising the said anode disk further comprises a stationary anode. The anode disk is positioned between the cathode of the x-ray source and the stationary anode. During operation, an electron beam passes the slit under an predefined beam angle different from the beam centre of the beam focus and hits the stationary anode or alternatively a stationary target. The stationary anode creates a secondary focus which may represent at least partially an amount of scattered radiation which could be detected. In one aspect of the embodiment, the beam angle respectively the beam angulation β and the distance h between the plane of the first focus spot area at the anode disk and the plane of the secondary focus spot area on the stationary anode are as such, that the offset Δd_{off} of the secondary focus relatively to the beam centre is larger than the focal range FR_{ASG} of the ASG:

Δd_{off}=(tan β)/h>FR_{ASG}

In another aspect of the embodiment, the beam size may be at minimum at the primary focus and may be increase up to a maximum at the secondary focal spot at the stationary anode.

According to another exemplary embodiment of the present invention, an examination apparatus for examination of an object of interest comprises the said x-ray tube, adapted for emitting electromagnetic radiation to an object of interest, a detector unit adapted for detecting image data and scatter data from the object of interest, and an anti-scatter-grid adapted for filtering the electromagnetic radiation. The anti-scatter-grid defining a focal range, from where a first focus could send radiation directly to the detector unit. The image data of a first focus of the electromagnetic radiation relative to the detector unit is detected for a first period of time. The scatter data of a secondary focus of the electromagnetic radiation is detected for a second period of time which is significantly smaller than the first period of time. The first focus is created by the anode disk of the x-ray tube. The secondary focus is created by the stationary anode of the x-ray tube.

Thus, the scatter data which is detected from the detector unit may comprise only little or even none direct radiation data.

According to another exemplary aspect of the invention, the anti-scatter-grid is a 1-dimensional anti-scatter-grid.

This may provide for an easy fabrication of the ASG. Furthermore, this may allow for the application for advanced CT system concepts such as stereo-tube design.

According to another aspect of the invention, the spatial angle of the first focus is smaller than the spatial angle of the secondary focus.

According to another exemplary aspect of the invention, the secondary focus is outside of the focal range of the anti scatter grid.

According to another exemplary aspect of the invention, the first period of time and the second period of time correspond to a detection sequence.

According to another exemplary aspect of the invention, the examination apparatus is configured as one of the group consisting of a baggage inspection apparatus, a medical application apparatus, a material testing apparatus and a material science analysis apparatus. A field of application of the invention may be material science analysis, since the defined functionality of the invention may allow for a secure, reliable and highly accurate analysis of a material.

According to another exemplary aspect of the invention, a method of examination of an object of interest, comprises the steps of: emitting electromagnetic radiation within a first focus by an anode disk of a rotating anode x-ray tube, emitting electromagnetic radiation within a secondary focus by a stationary anode of the x-ray tube, detecting image data from the first focus for a first period of time, detecting scatter data of a secondary focus for a second period of time. The stationary anode may simply be the metallic frame of the tube, which may be covered with an X-ray generating conductive material. It may be cooled by a fluid.

According to another exemplary aspect of the invention, the second period of time is in a range between 5 μs and 40 μs.

According to another exemplary aspect of the invention, a computer-readable medium, in which a computer program of examination of an object of interest is stored which, when being executed by a processor, is adapted to carry out the steps of emitting electromagnetic radiation within a first focus by an anode disk of a rotating anode x-ray tube, emitting electromagnetic radiation within a secondary focus by a stationary anode of the x-ray tube, detecting image data from the first focus for a first period of time, and detecting scatter data of a secondary focus for a second period of time.

According to another exemplary aspect of the invention, a program element of examination of an object of interest, which, when being executed by a processor, is adapted to carry out the steps of emitting electromagnetic radiation within a first focus by an anode disk of a rotating anode x-ray tube, emitting electromagnetic radiation within a secondary focus by a stationary anode of the x-ray tube, detecting image data from the first focus for a first period of time, and detecting scatter data of a secondary focus for a second period of time.

The program element may preferably be loaded into working memories of a data processor. The data processor may thus be equipped to carry out exemplary embodiments of the methods of the present invention. The computer program may be written in any suitable programming language, such as, for example, C++ and may be stored on a computer-readable medium, such as a CD-ROM. Also, the computer program may be available from a network, such as the WorldWideWeb, from which it may be downloaded into image processing units or processors, or any suitable computers.

According to the aforesaid, the scatter data may only comprise a relatively small amount of direct radiation. Thus, during the said second period of time, in other words, during scatter measurement the amount of direct radiation reaching the detector may be significantly reduced. Therefore, the resulting measurement (scatter data) may basically only contain scattered photons. Such a measurement may provide a good estimation of the scatter contribution to the imaging measurements.

According to a further aspect of the present invention, a detection sequence may be pre-determined. Then, during data acquisition, the focal spot is switched mechanically between the first position at the anode disk and the second position at the stationary anode (for acquisition of image data and scatter data, respectively) according to the pre-determined detection or switching sequence. Since scatter may vary slowly in the spatial domain, the scatter measurements may only sporadically be interleaved in the image acquisition (according to the pre-determined detection sequence).

According to another aspect of the invention a cost effective x-ray tube for both a so-called circle acquisition and a so-called line acquisition is claimed. Preferably, the x-ray tube may be used for Axial Cone Beam Computer Tomography. The circle acquisition takes place in an axial CT mode, wherein the gantry of the CT apparatus is rotating around a rotational axis, while the object of interest, i.e. a patient, rests on a patient table or is displaced along a direction parallel to the rotational axis of the gantry (helical scan). During the circle acquisition, moving objects may be measured, i.e. the patients beating heart.

During line acquisition cone beam artefact generating material may be measured by the CT apparatus. In an exemplary embodiment according to the said aspect a stationary anode is used during line acquisition. In a further exemplary embodiment according to the said aspect a second disk anode may be used. The stationary anode or the second disc anode is moved such that the distance between the cathode and a focal spot at the stationary anode or the second disc anode is increased or decreased. Due to the changing distance of the electron beam flying through the slit and hitting the focal spot

area on the stationary anode or on the second disc anode beneath, the resulting x-ray beam is shifted with regard to the slit anode disk. In one embodiment the movement of the stationary anode or the second disk anode may be oriented in an axial direction of a rotational axis of the slitted disk anode. Further, the movement of the stationary anode or, alternatively, the second disk anode may be carried out in another direction with a small angle of a few degrees spanned between the said axial direction and the movement direction. The said angle may be selected from a range between 0° to 40°.

Alternatively, the orientation of the movement of the stationary anode or the second disk anode may be in a direction perpendicular to said rotational axis of the slitted disk anode. Further the movement of the stationary cathode or, alternatively, the second disk anode may be carried out with a small angle of a few degrees spanned between a normal to the said axial direction and the movement direction. Thus, the said angle may be selected from a range between 0° to 40°.

Further, the a stationary anode or the second disk anode may be moved longitudinal and lateral for enhancing the anode power rating. According to an other embodiment of the invention, the effective anode angle of the stationary anode or the second rotating disk anode can be changed during a linear motion of the anode. Thus, mechanical tilting and re-focusing of the electron-beam is enabled. The motion of the stationary anode or the second disk anode may be achieved continuously. This mechanical adjustment of the anode angle during motion may reduce specific heat loading while sufficient detector coverage via the x-ray beam is maintained.

According to a further aspect the x-ray tube comprises an aperture device wherein the aperture device is movable at least in axial direction of the cathode electron beam. The moving aperture enables to block the emitting x-ray beam of the anode disk during the line acquisition or to block the emitting x-ray beam of the stationary anode or the second disk anode during the centre acquisition. Preferably, the aperture movement is carried out in the direction of the rotational axis of the gantry (z-direction) or in direction of the rotational axis of the slitted disk anode which direction is may be the same as the direction of the cathode electron beam.

According to another aspect of the invention the x-ray tube may comprises a volume changing device containing the movable stationary anode or a second disc anode, wherein the volume changing device is adapted to maintain the vacuum of the x-ray tube. In one embodiment the volume changing device is a bellows device.

According to yet another aspect, the movement of the stationary anode or the second disk anode may be carried out by a linear actuator. Further, the said movement may be carried out by moving the anode around a pivot point. Further, the movement of the anode and the movement of the aperture device may be mechanically linked by an adequate linking device.

One preferred method of examination of an object of interest, method comprises the steps of emitting electromagnetic radiation, i.e. x-radiation, within a first focus by an slitted anode disk of a rotating anode x-ray tube, emitting electromagnetic radiation within a secondary focus by a stationary anode or second rotating disk anode of the x-ray tube and detecting image data from the first focus for a first period of time;

detecting scatter data or image data of a secondary focus for a second period of time.

In one aspect of the said method, the secondary focus moves along a direction of the electron beam during a first sequence of multiple 2^{nd} (second) periods of time (line acquisition).

In one embodiment, the method further comprises the step of blocking the emitting electromagnetic radiation of the stationary anode or the second rotating disk anode during a second sequence of multiple first periods of time.

According to yet another aspect of the invention, the emitted electromagnetic radiation of the cathode may be modulated by high voltage pulsing such, that the maximum of cathode beam energy is reached if the cathode beam passes one slot of the slitted anode disk. The modulation may help to reduce power loading of the rotating anode disk.

According to a further aspect of the invention, the high voltage for emitting electromagnetic radiation of the cathode is raised during the first emitting sequence (line acquisition) compared to the second sequence (circle acquisition). Thus, the contrast resolution of the images may be improved, because high-contrast objects, which create cone beam artefacts are measured preferably during the line acquisition mode.

In another aspect of the invention, any shape of the motion track versus time of the stationary anode or the second disk anode is possible during the line acquisition. Thus, the motion track may be at least partly linear, sinusoidal or triangular to optimize the image data density versus the z-direction.

It should be noted that in yet another aspect the line acquisition may be carried out as long as needed to gather sufficient photons at a detector.

These and other aspects of the present invention will become apparent from and elucidated with reference to the embodiments described hereinafter.

Exemplary embodiments of the present invention will be described in the following, with reference to the following drawings.

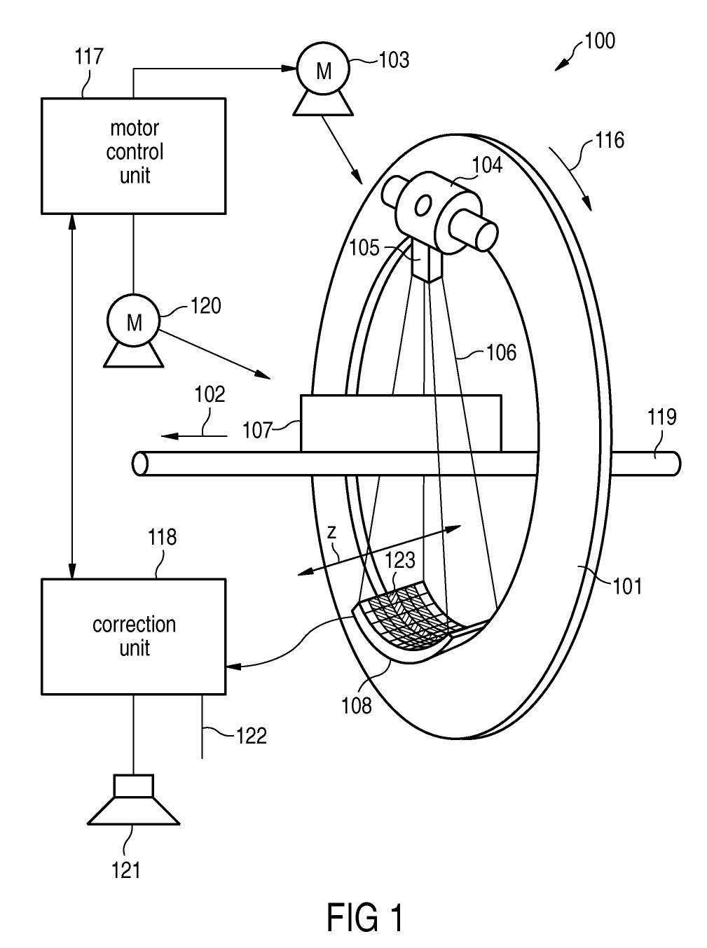

FIG. 1 shows a simplified schematic representation of an examination apparatus according to an exemplary embodiment of the present invention.

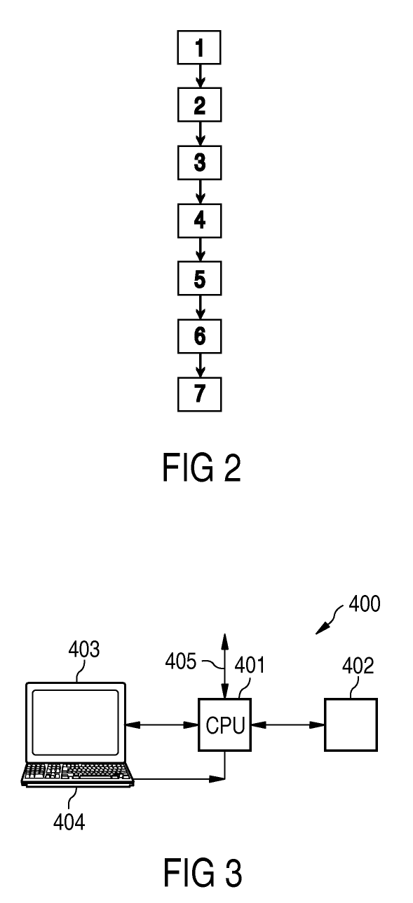

FIG. 2 shows a flow-chart of an exemplary method according to the present invention.

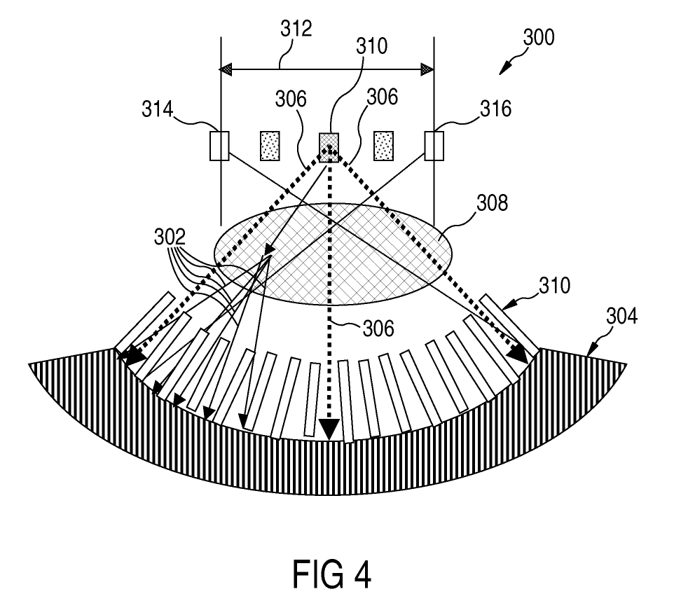

FIG. 3 shows an exemplary embodiment of an image processing device according to the present invention, for executing an exemplary embodiment of a method in accordance with the present invention.

FIG. 4 shows the beam geometry of a tomography apparatus.

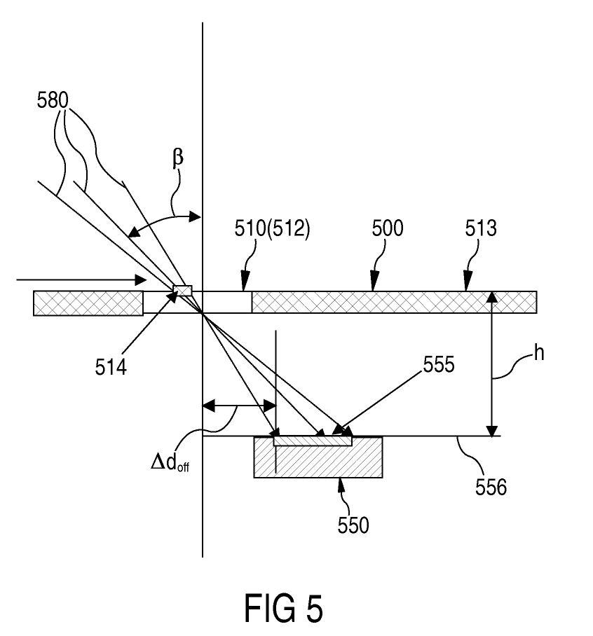

FIG. 5 shows in a sectional side view an anode arrangement with a anode disk and a stationary anode.

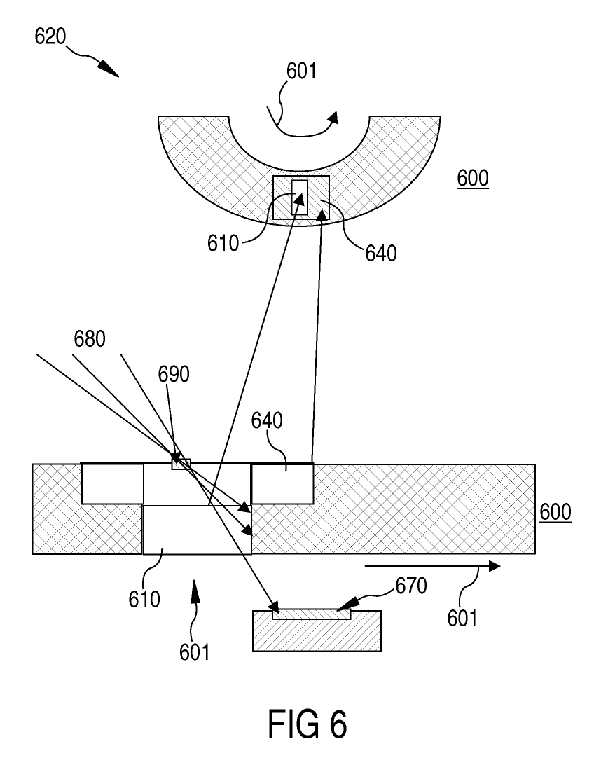

FIG. 6 shows schematically another embodiment of an anode disk.

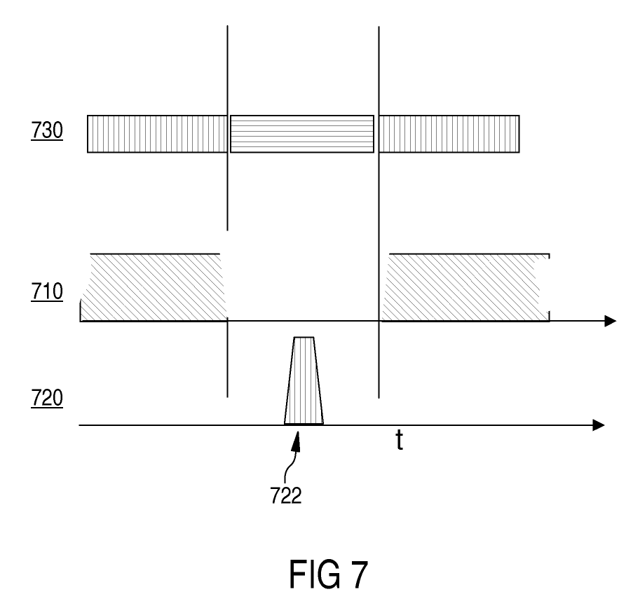

FIG. 7 shows in a diagram the amount of x-rays.

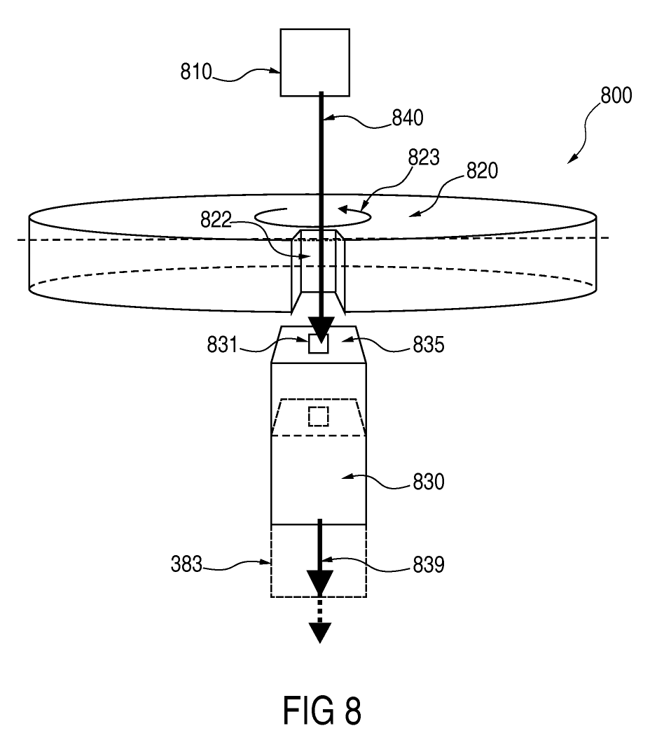

FIG. 8 shows a cathode, a rotating disk anode with a slit and a moving stationary anode.

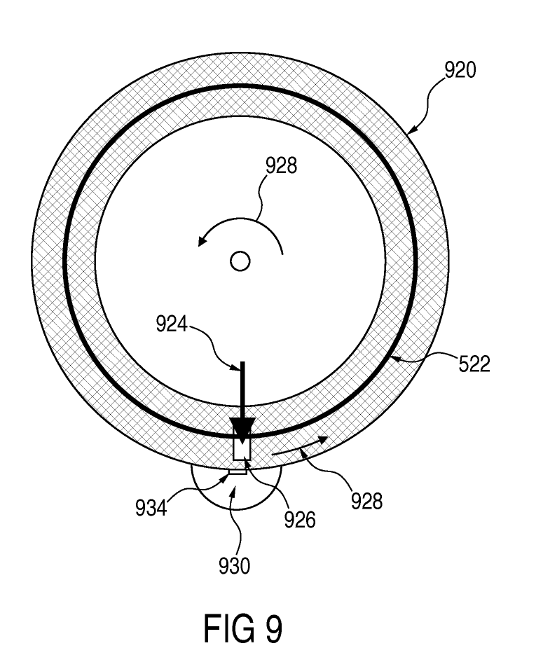

FIG. 9 shows a top view of the rotating anode with the moving stationary anode.

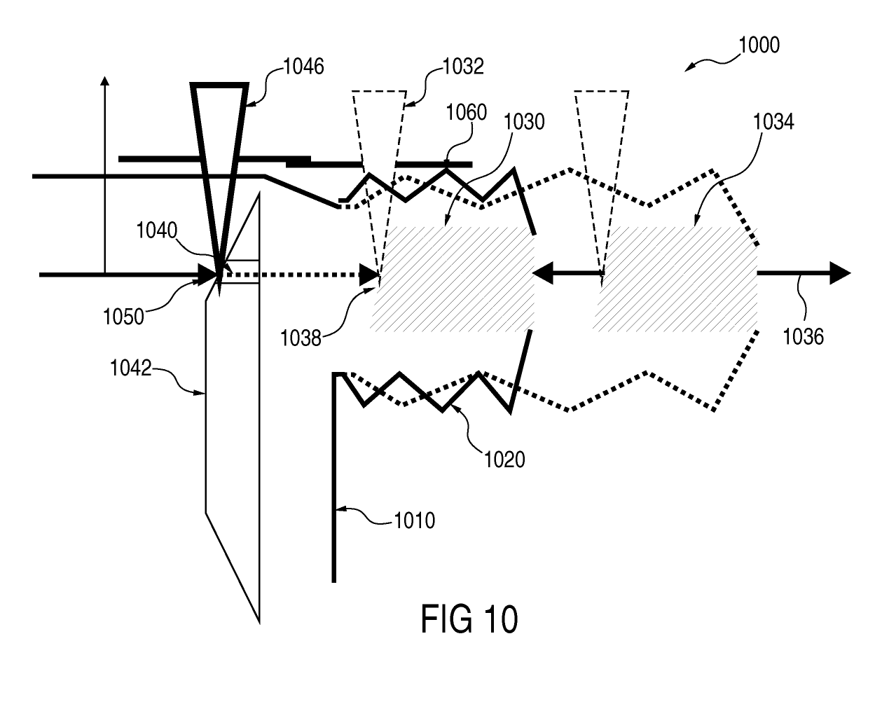

FIG. 10 shows a partial side view of a tube envelope with the rotating anode and the moving stationary anode in a bellows system.

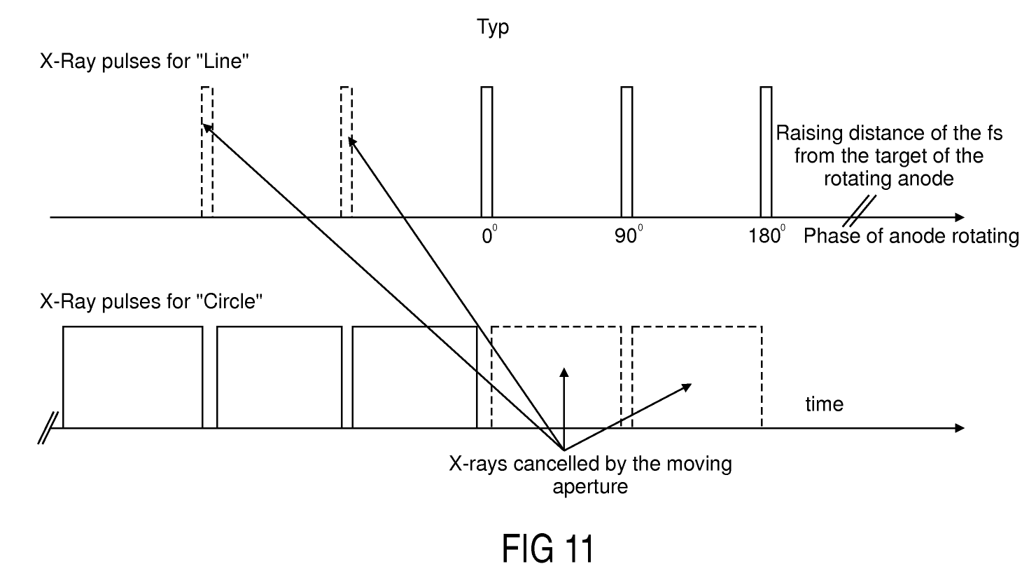

FIG. 11 shows a pulsing scheme of two different pulsing modes (line acquisition and circle acquisition), and

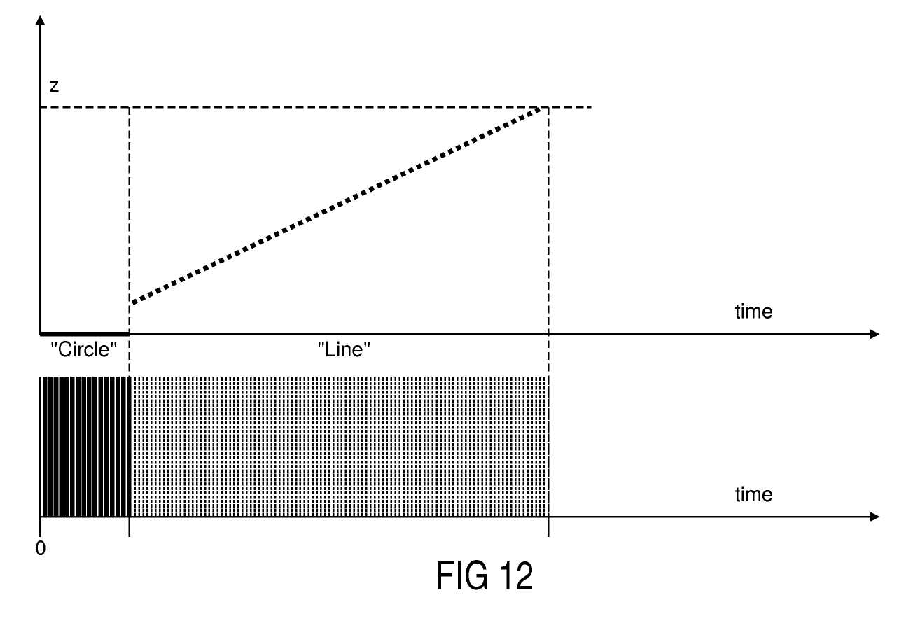

FIG. 12 shows a further pulsing scheme together with a focal spot position of the moving stationary anode.

The illustration in the drawings is schematically. In different drawings, similar or identical elements are provided with the same reference numerals.

FIG. 1 shows an examination apparatus according to an exemplary embodiment of the present invention which is adapted as a computer tomography apparatus. With reference to this exemplary embodiment, the present invention will be described for the application in medical imaging. However, it should be noted that the present invention is not limited to this application, but may also be applied in the field of baggage inspection, or other industrial applications, such as material testing.

The computer tomography apparatus 100 depicted in FIG. 1 is a cone-beam CT scanner. The CT scanner depicted in FIG. 1 comprises a gantry 101, which is rotatable around a rotational axis 102. The gantry 101 is driven by means of a motor 103. Reference numeral 104 designates a source of radiation such as an X-ray source.

Reference numeral 105 designates an aperture system which forms the radiation beam emitted from the radiation source to a cone-shaped radiation beam 106. The cone-beam 106 is directed such that it penetrates an object of interest 107 arranged in the centre of the gantry 101, i.e. in an examination region of the CT scanner, and impinges onto the detector 108. As may be taken from FIG. 1, the detector 108 is arranged on the gantry 101 opposite to the source of radiation 104, such that the surface of the detector 108 is covered by the cone-beam 106. The detector 108, which is depicted in FIG. 1, comprises a plurality of detector elements 123 each capable of detecting, in a spatial resolving manner, X-rays or individual photons which have penetrated the object of interest 107.

During a scan of the object of interest 107, the source of radiation 104, the aperture system 105 and the detector 108 are rotated along the gantry 101 in the direction indicated by arrow 116. For rotation of the gantry 101 with the source of radiation 104, the aperture system 105 and the detector 108, the motor 103 is connected to a motor control unit 117, which is connected to a calculation or correction unit 118.

In FIG. 1, the object of interest 107 may be a patient or an item of baggage which is disposed on a conveyor belt 119. During the scan of the object of interest 107, while the gantry 101 rotates around the item of baggage 107, the conveyor belt 119 displaces the object of interest 107 along a direction parallel to the rotational axis 102 of the gantry 101. By this, the object of interest 107 is scanned along a helical scan path. The conveyor belt 119 may also be stopped during the scans to thereby measure single slices. Instead of providing a conveyor belt 119, for example, in medical applications where the object of interest 107 is a patient, a movable table may be used. However, it should be noted that in all of the described cases it may also be possible to perform other scan paths such as the saddle trajectory by moving the table periodically back and forth at twice the frequency of the source-detector arrangement.

The detector 108 may be connected to the calculation or correction unit 118. The correction unit 118 may receive the detection result, i.e. the read-outs from the detector elements 123 of the detector 108 and may determine a scanning result on the basis of the read-outs. Furthermore, the correction unit 118 communicates with the motor control unit 117 in order to coordinate the movement of the gantry 101 with motors 103 and 120 with the conveyor belt 119.

The correction unit 118 may be adapted for correcting image data on the basis of scatter data, wherein the image data is detected during a first period of time and the scatter data is detected during a second period of time, according to aspect of the present invention.

The correction unit 118 may be realized by a data processor to process read-outs from the detector elements 123 of the detector 108.

Furthermore, as may be taken from FIG. 1, the correction unit 118 may be connected to a loudspeaker 121, for example, to automatically output an alarm in case of the detection of suspicious material in the item of baggage 107.

The computer tomography apparatus 100 for examination of the object of interest 107 includes the detector 108 having the plurality of detecting elements under an anti-scatter-grid (ASG) 123 arranged in a matrix-like manner, each being adapted to detect X-rays. Furthermore, the computer tomography apparatus 100 comprises the determination unit or reconstruction unit 118 adapted for reconstructing an image of the object of interest 107.

The computer tomography apparatus 100 comprises the X-ray source 104 adapted to emit X-rays to the object of interest 107. The collimator 105 provided between the electromagnetic radiation source 104 and the detecting elements under the anti-scatter-grid (ASG) 123 is adapted to collimate an electromagnetic radiation beam emitted from the electromagnetic radiation source 104 to form a cone-beam. The detecting elements under the anti-scatter-grid (ASG) 123 form a multi-slice detector array 108. The computer tomography apparatus 100 may be configured as a medical imaging apparatus or baggage inspection apparatus.

FIG. 2 shows a flow-chart of an exemplary method according to the present invention for directly measuring the scatter radiation during one rotation of an anode disk, not shown here, and for using this measurement for a correction of the contaminated image data. The method starts at step 1 with the emission of electromagnetic radiation within a first focus by an anode disk of a rotating anode x-ray tube. Furthermore, a conventional CT scan is performed.

In step 2, image data from the first focus for a first period of time were detected;

Then, in step 3, the conventional data acquisition is interleaved with a scatter measurement by rotating the anode disk such that the electron beam penetrates a slit of the anode disk and hits a stationary anode and, thus electromagnetic radiation within a secondary focus by the stationary anode of the x-ray tube is created. In other words, for a short period of time, a secondary focal spot is created outside of a focal range of the ASG, which allows to measure scattered radiation during one of the integration periods of the CT system. The secondary focal spot radiates synchronically to the rotational speed of the anode disk, which is e.g. 100 m/s.

Furthermore, in step 4, scatter data of a secondary focus for a second period of time is detected. The measuring of scatter data is performed by utilizing, for example, 1-dimensional anti-scatter-grid and an X-ray tube with an electronic focal spot movement.

The conventional 1-dimensional anti-scatter-grid may have anti-scatter-lamella along the Z-direction to reduce the scatter in the fan direction.

Then, in step 5, a low-pass filtering may be performed on the scatter measurements.

After that, the image data may be corrected on the basis of the scatter data by a correction unit. This correction may be performed by subtracting the scatter measurements from the imaging measurements to generate a corrected projection.

Then, in step 7, a reconstruction may be performed with the corrected projections, resulting in a corrected image of the object of interest.

The invention makes use of the fact that scatter radiation may usually have only very small spatial variations. A relatively small movement of the focal spot from a position at the anode disk to a position of the stationary anode for a scatter measurement may have very little impact on the scatter compared to the imaging measurements. Therefore, the imaging measurements may be interleaved with scatter measurements. Since scatter may very slowly in the spatial domain, the scatter measurements may sporadically be interleaved in the image acquisition (for example according to a pre-determined sequence.

FIG. 3 depicts an exemplary embodiment of a data processing device 400 for executing a method in accordance with the present invention. The data processing device 400 depicted in FIG. 3 comprises a central processing unit (CPU) or image processor 401 connected to a memory 402 for storing an image depicting an object of interest, such as a patient or an item of baggage. The data processor 401 may be connected to a plurality of input/output network or diagnosis devices, such as a CT device. The data processor 401 may furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 3.

Furthermore, via the bus system 405, it may also be possible to connect the image processing and control processor 401 to, for example, a motion monitor, which monitors a motion of the object of interest. In case, for example, a lung of a patient is imaged, the motion sensor may be an exhalation sensor. In case the heart is imaged, the motion sensor may be an electrocardiogram.

FIG. 4 shows schematically the beam geometry of a tomography apparatus 300. Arrows indicate scattered radiation 302 generated by an object of interest 308. The scattered radiation 302 detoriates image signals especially for large detectors 304, as it overlaps with direct radiation 306. An anti-scatter-grid (ASG) 310 limits the space angle, from which scattered radiation may reach detector cells of the detector unit 304 and therefore improves the ration of scattered radiation 302 to direct radiation 306. For measurement of the scattered radiation 302, a focal spot 310 of an anode would need to be moved out of the ASG focal range 312 FR_{ASG} to a position 314 or 316. The ASG focal range is defined as the range from where the focal spot could send direct radiation 306 to the detector unit 304.

FIG. 5 shows in a schematically sectional side view an anode arrangement with a anode disk 500 and a stationary anode 550. The anode disk 500 comprises a slit 510 which is positioned within a circular target area 512 of the anode disk 500 and has a width of 2 mm. The slit opening is adapted to be penetrated at least partially by an electron beam 580 of a cathode of the x-ray tube, not shown here during a time period of 20 μs if the anode track speed is 100 m/s. Thus, the time period of the passage through the slit 510 is short enough to save the target 550 in the secondary focus 555 from melting, even under full beam power.

When the electron beam 580 passes through the slit 510 under an angle β, it hits the stationary anode 550 and creates a secondary focal spot 555.

The beam angle β and the distance h between a plane 513 of an area of a first focal spot 514 at the anode disk 510 and the plane 556 of the area the secondary focus spot 555 on the stationary anode 550 are as such, that the offset Δd_{off} of the secondary focus relatively to the beam centre is larger than the focal range FR_{ASG} of the ASG:

Δd_{off}=(tan Δ)/h>FR_{ASG} 312. As the beam size of beam 580 is minimal at the first focus 514, the secondary focus 555 is usually larger. Undesired transition effects are being avoided by a proper shape of the slit. The offset between the secondary focus 555 and the first focus 514 exists usually also in azimuth direction. Thus the arrangement may be generally applicable for both, a one-dimesional and a two-dimensional ASG.

FIG. 6 shows schematically another embodiment of an anode disk 600 rotating in direction 601 indicated by an arrow in a topview section 620 and a partial sectional side view 630. The anode disk comprises a slit 610 which is arranged in a notch 640. Also shown is the beam direction of a beam 680, indicated by arrows and the positions of a first focus 690 and a secondary focus 670. The notch or cavity 640 may help to avoid transition effects when the electron beam passes the opening 601 of the anode disk 600. An undesired transition effect would be the co-existence of two X-ray emitting areas (“focal spots”) at the same time.

FIG. 7 shows in a schematically diagram the amount 710 of x-rays created from the first focus of the anode disk and the amount 720 of x-rays created from the secondary focus of the anode disk shown in FIG. 5 during one CT integration period 730 over time t. One pulse 722 is generated from the stationary anode per revolution of the rotating anode disk.

FIG. 8 shows an X-ray tube arrangement 800 with cathode 810, a rotating disk anode 820 with one slit 822 and a moving stationary anode 830. The rotating direction 823 is indicated by an arrow. An arrow indicates the electron beam 840 emitted by the cathode 810. The electron beam 840 passes through the slit 822 and impinges at X-ray focal spot 831 of a target surface 835 of the stationary anode 830 during a line acquisition mode. During said line acquisition mode an actuator (not shown here) moves the stationary anode 830 in an axial direction (z-direction of FIG. 1). The axial moving direction 839 is indicated by an solid arrow. Further, the stationary anode 830 is shown in a further (future) position 838. The distance between the focal spot shown with a solid line and the focal spot in the future position (dashed line) will be obtained after several intermediate measurements of the emitted x-rays of the stationary anode 830.

FIG. 9 shows a top view of a rotating disk anode 920 with the moving stationary anode 930 beneath. A black circle indicates the track 922 of an electron beam 924, shown as an arrow. Further a slit 926 is shown. Two arrows indicate the rotating direction 928 of the disk anode 920. The electron beam is emitted from a cathode (not shown) and flies from the top through the slit 926 and hits the focal spot 934 of the stationary anode 930.

FIG. 10 shows a partial side view of an x-ray tube 1000 with a partially shown tube envelope 1010. Further, the x-ray tube 1000 comprises a volume changing device 1020 in form of a bellows system. The bellows system contains the movable stationary anode 1030, and is adapted to maintain the vacuum of the x-ray tube 1000. Further the bellows system contains a linear actuator, not shown here. An electron beam 1050 flies from a cathode (not shown) through the slit 1040 of the rotating disk anode 1042 and hits the focal spot 1038 of the stationary anode 1030 beneath. The slit chops the electronbeam and lets it pass in pulses towards the stationary anode. The electron beam pulses are short compared to the rotation period of the rotating anode. The resulting x-ray beam 1032 is indicated by a dashed triangle. A further intermediate position 1034 of the stationary anode is shown on the left side of FIG. 10. This position is reached during the line acquisition via an axial movement 1036. During the circle acquisition the resulting x-ray beam 1046 is emitted b the disk anode 1042. An arrow indicates the constant distance r from the focal spot of the stationary anode which is in line with the electron beam 1050 to a detector device (not shown). 7. An aperture device 1060 is movable at least in axial direction of the cathode electron beam to block the emitting x-radiation of the anode disk during a first sequence (line acquisition).

FIG. 11 shows a pulsing scheme of two different pulsing modes (line acquisition and circle acquisition). During the circle acquisition an object is measured in an axial CT mode (gantry rotating, patient at rest). The line acquisition is used to i.e. to measure cone beam artifact generating material by moving the X-ray source in i.e. z-direction parallel to the Region of Interest. For this measurement, the temporal and the spatial resolution in x-direction is not critical. The resolution in z-direction and the focal spot distance to the detector must both stay constant due to the focus of the ASG. In the upper half of FIG. 11 X-ray pulses during line acquisition are shown. Note, that an anode disk with four slits was used in this embodiment. Thus, every 90° a pulse of 20 μs length is impinged at the focal spot of the moving stationary anode. With time proceeding the distance raised between the focal spot of the stationary (non rotating) anode and the focal spot of the disk anode (upper half of FIG. 12). In the lower half of FIG. 11X-ray pulses during circle acquisition are shown. Note, that an anode disk with Pulses of 1250 μs is length each are impinged with a frequency of 800 Hz at the focal spot track of the disk anode. With time proceeding the distance raised between the focal spot of the stationary (non rotating) anode and the focal spot of the disk anode (upper half of FIG. 12). Note the first two X-ray pulses in the upper half and the last two X-ray pulses in the lower half (Dashed lines) are cancelled due to the moving aperture (FIG. 10).

FIG. 12 shows a further pulsing scheme (lower half) together with a focal spot position (upper half) of the moving stationary anode. In the first 0.15 seconds the circe acquisition mode is applied. From 0.15 s to 0.9 s the line acquisition mode is applied with its short pulse length.

The raising of distance during the line acquisition is shown in the upper half of FIG. 12. At the distance versus time diagram the focal spot of the disk anode is at 0 cm in the circle acquisition mode in the first 150 ms (solid bold line). The focal spot is switched to 2 cm due to the changing of X-ray blocking/passage by the moving aperture.

A method for the examination of an object of interest, is shown in FIG. 2. In a first step 1 electromagnetic radiation within a first focus by an anode disk of a rotating anode x-ray tube is emitted. In step 2 electromagnetic radiation within a secondary focus by a stationary anode or second rotating disk anode of the x-ray tube is emitted.

During this steps image data from the first focus for a first period of time (i.e. 1250 μs in FIG. 11) and image data of a secondary focus for a second period of time (i.e. 20 μs in FIG. 11) is generated. The secondary focus moves along a direction of the a cathode electron beam during a first sequence (0.75 s) of multiple second periods of time (20 μs in FIG. 11), the so-called line acquisition. During this movement, the emitting electromagnetic radiation of the anode disk is blocked by a movable aperture. After or before line acquisition takes place, the emitted electromagnetic radiation of the stationary anode or the second rotating disk anode during a second sequence (circle acquisition, i.e. 0.15 s) of multiple first periods of time is blocked by the moving aperture.

It should be noted that the term “comprising” does not exclude other elements or steps and the “a” or “an” does not exclude a plurality. Also elements described in association with different embodiments may be combined. The further steps 3 to 7 may relate to further acquisition steps and image pre-processing.

It should also be noted that reference signs in the claims shall not be construed as limiting the scope of the claims.

1. An anode disk, for measuring scattered radiation of a rotating anode x-ray tube or for emitting electromagnetic radiation to an object of interest, the anode disk comprising:
at least one slit;
wherein the slit is positioned in a target area of the anode disk; and wherein the slit opening is adapted to be penetrated at least partially by an electron beam of a cathode of the x-ray tube.

2. The anode disk of claim 1; wherein the width of the slit is in a range between 0.9 mm and 10 mm or between 0.4 mm and 30 mm.

3. The anode disk according to claim 1; the anode disk further comprising
at least one cavity;
wherein the cavity incorporates the slit.

4. An x-ray tube, comprising the anode disk according to claim 1; the x-ray tube further comprising:
a stationary anode or a second rotating disk anode;
a cathode;
wherein the anode disk is positioned between the cathode and the stationary anode or the second rotating disk anode.

5. The x-ray tube of claim 4, wherein the stationary anode or the second disc anode is movable such, that the distance between the cathode and a focal spot at the stationary anode or the second disc anode is changeable.

6. The x-ray tube of claim 4, wherein the stationary anode or the second disc anode is movable in a direction, parallel and/or perpendicular to the axial direction of the cathode electron beam.

7. The x-ray tube of claim 4, comprising
an aperture device, wherein the aperture device is movable at least in axial direction of the cathode electron beam.

8. The x-ray tube of claim 4, comprising
a volume changing device containing the movable stationary anode or a second disc anode, wherein the volume changing device is adapted to maintain the vacuum of the x-ray tube.

9. The x-ray tube of claim 7, wherein the volume changing device is a bellows device.

10. An examination apparatus for examination of an object of interest, the examination apparatus comprising:
the x-ray tube of claim 4, adapted for emitting electromagnetic radiation to the object of interest;
a detector unit adapted for detecting image data and/or scatter data from the object of interest;
an anti-scatter-grid adapted for attenuating the electromagnetic radiation;
the anti-scatter-grid defining a focal range, from where a first focus could send radiation directly to the detector unit;
wherein the image data of a first focus of the electromagnetic radiation relative to the detector unit is detected for a first period of time; and
wherein the scatter data or image data of a secondary focus of the electromagnetic radiation is detected for a second period of time which is significantly smaller than the first period of time;
wherein the first focus is created by the anode disk of the x-ray tube.
wherein the secondary focus is created by the stationary anode or the second anode disk of the x-ray tube.

11. The examination apparatus of claim 10,
wherein the secondary focus is outside of the focal range of the anti scatter grid.

12. The examination apparatus according to claim 10,
wherein the anti-scatter-grid (ASG) is a one-dimensional anti-scatter-grid or a two-dimensional anti-scatter-grid.

13. The examination apparatus according to claim 10,
wherein the first period of time and the second period of time correspond to a detection sequence.

14. The examination apparatus according to claim 10, configured as one of the group consisting of a baggage inspection apparatus, a medical application apparatus, a material testing apparatus and a material science analysis apparatus.

15. A method of examination of an object of interest, method comprising the steps of:
emitting electromagnetic radiation within a first focus by an anode disk of a rotating anode x-ray tube;
emitting electromagnetic radiation within a secondary focus by a stationary anode or second rotating disk anode of the x-ray tube;
detecting image data from the first focus for a first period of time;
detecting scatter data or image data of a secondary focus for a second period of time.

16. The method of claim 15, wherein the secondary focus moves along a direction of a cathode electron beam during a first sequence of multiple second periods of time.

17. The method of claim 15, comprising the step
moving the stationary anode or the second disc anode such, that the distance between the cathode and a focal spot at the stationary anode or the second disc anode is changeable.

18. The method of claim 15, wherein the method further comprising the step
blocking the emitting electromagnetic radiation of the anode disk during the first sequence.

19. The method of claim 15, wherein the method further comprising the step
blocking the emitting electromagnetic radiation of the stationary anode or the second rotating disk anode during a second sequence of multiple first periods of time.

20. The method of claim 10; wherein the second period of time is in a range between 5 μs and 200 μs or between 200 μs and 2000 μs.

21. A computer-readable medium, in which a computer program of examination of an object of interest is stored which, when being executed by a processor, is adapted to carry out the steps of:
emitting electromagnetic radiation within a first focus by an anode disk of a rotating anode x-ray tube;
emitting electromagnetic radiation within a secondary focus by a stationary anode or second anode disk of the x-ray tube;
detecting image data from the first focus for a first period of time;
detecting scatter data or image date of a secondary focus for a second period of time.

22. A program element of examination of an object of interest, which, when being executed by a processor, is adapted to carry out the steps of:
emitting electromagnetic radiation within a first focus by an anode disk of a rotating anode x-ray tube;
emitting electromagnetic radiation within a secondary focus by a stationary anode or a second rotating anode disk of the x-ray tube;
detecting image data from the first focus for a first period of time;
detecting scatter data or image data of a secondary focus for a second period of time.
